(19) United States
(12) Patent Application Publication
(10) Pub. No.: US 2004/0260331 A1
(43) Pub. Date: Dec. 23, 2004

(54) Beta titanium embolic protection frame and guide wire

(76) Inventors: Peter D'Aquanni, Murrieta, CA (US); Wayne E. Cornish, Fallbrook, CA (US); Mark T. Richardson, Escondido, CA (US); Ryan Grandfield, Murrieta, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/600,688

(22) Filed: Jun. 20, 2003

(51) Int. Cl. A61M 29/00

(52) U.S. Cl. 606/200

(57) ABSTRACT

An expandable frame for use in conjunction with an embolic filtering device includes a Beta titanium alloy, and more particularly, a Beta III titanium alloy. The expandable frame may be coupled to the distal end of a guide wire. The guide wire may also include Beta III titanium. The Beta III titanium material can be processed to have an elastic modulus about twice that of other expandable frame or guide wire materials, thereby allowing a reduction of the cross-sectional area of the frame and/or guide wire while maintaining the bending stiffness of the frame and/or guide wire. The Beta III titanium also improves manufacturability of the frames, thereby reducing manufacturing costs.

### BACKGROUND OF THE INVENTION

The present invention relates generally to medical devices including filtering devices and guide wires. Regarding the former, the filtering devices may be part of systems which can be used when an interventional procedure is being performed in a stenosed or occluded region of a body vessel to capture embolic material that may be created and released into the vessel during the procedure. The present invention is more particularly directed to an embolic filtering device made with a self-expanding frame (also referred to as a basket or cage) having good flexibility and bendability to reach often tortuous areas of treatment. The present invention is particularly useful when an interventional procedure, such as balloon angioplasty, stenting procedure, laser angioplasty or atherectomy, is being performed in a critical body vessel, such as the carotid arteries, where the release of embolic debris into the bloodstream can occlude the flow of oxygenated blood to the brain, resulting in grave consequences to the patient. While the present invention is particularly useful in carotid procedures, the invention can be used in conjunction with any vascular interventional procedure in which an embolic risk is present.

Numerous procedures have been developed for treating occluded blood vessels to allow blood to flow without obstruction. Such procedures usually involve the percutaneous introduction of an interventional device into the lumen of the artery, usually by a catheter. One widely known and medically accepted procedure is balloon angioplasty in which an inflatable balloon is introduced within the stenosed region of the blood vessel to dilate the occluded vessel. The balloon dilatation catheter is initially inserted into the patient's arterial system and is advanced and manipulated into the area of stenosis in the artery. The balloon is inflated to compress the plaque and press the vessel wall radially outward to increase the diameter of the blood vessel, resulting in increased blood flow. The balloon is then deflated to a small profile so that the dilatation catheter can be withdrawn from the patient's vasculature and the blood flow resumed through the dilated artery. As should be appreciated by those skilled in the art, while the above-described procedure is typical, it is not the only method used in angioplasty.

Another procedure is laser angioplasty which utilizes a laser to ablate the stenosis by super heating and vaporizing the deposited plaque. Atherectomy is yet another method of treating a stenosed body vessel in which cutting blades are rotated to shave the deposited plaque from the arterial wall. A catheter is usually used to capture the shaved plaque or thrombus from the bloodstream during the procedure.

In the procedures of the kind referenced above, abrupt reclosure may occur or restenosis of the artery may develop over time, which may require another angioplasty procedure, a surgical bypass operation, or some other method of repairing or strengthening the area. To reduce the likelihood of the occurrence of abrupt reclosure and to strengthen the area, a physician can implant an intravascular prosthesis, commonly known as a stent, for maintaining vascular patency inside the artery across the lesion. The stent can be crimped tightly onto the balloon portion of the catheter and transported in its delivery diameter through the patient's vasculature. At the deployment site, the stent is expanded to a larger diameter, often by inflating the balloon portion of the catheter.

The above non-surgical interventional procedures, when successful, avoid the necessity of major surgical operations. However, there is one common problem which can become associated with all of these non-surgical procedures, namely, the potential release of embolic debris into the bloodstream that can occlude distal vasculature and cause significant health problems to the patient. For example, during deployment of a stent, it is possible that the metal struts of the stent can cut into the stenosis and shear off pieces of plaque that can travel downstream and lodge somewhere in the patient's vascular system. Pieces of plaque material are sometimes generated and become released into the bloodstream during a balloon angioplasty procedure. This is particularly true when the procedure is performed in a saphenous vein graft (SVG). Additionally, while complete vaporization of plaque is the intended goal during laser angioplasty, sometimes particles are not fully vaporized and enter the bloodstream. Likewise, not all of the emboli created during an atherectomy procedure may be drawn into the catheter and, as a result, may enter the bloodstream as well.

When any of the above-described procedures are performed in the carotid arteries, the release of emboli into the circulatory system can be extremely dangerous and sometimes fatal to the patient. Debris carried by the bloodstream to distal vessels of the brain can cause cerebral vessels to occlude, resulting in a stroke, and in some cases, death. Therefore, although cerebral percutaneous transluminal angioplasty has been performed in the past, the number of procedures performed has been somewhat limited due to the justifiable fear of an embolic stroke occurring should embolic debris enter the bloodstream and block vital downstream blood passages.

Medical devices have been developed to attempt to deal with the problem created when debris or fragments enter the circulatory system following vessel treatment utilizing any one of the above-identified procedures. One approach which has been attempted is the cutting of any debris into minute sizes which pose little chance of becoming occluded in major vessels within the patient's vasculature. However, it is often difficult to control the size of the fragments which are formed, and the potential risk of vessel occlusion still exists, making such a procedure in the carotid arteries a high-risk proposition.

Other techniques include the use of catheters with a vacuum source which provides temporary suction to remove embolic debris from the bloodstream. However, there can be complications associated with such systems if the vacuum catheter does not remove all of the embolic material from the bloodstream. Also, a powerful suction could cause trauma to the patient's vasculature.

Another technique which has had some success utilizes a filter or trap downstream from the treatment site to capture embolic debris before it reaches the smaller blood vessels downstream. The placement of a filter in the patient's vasculature during treatment of the vascular lesion can reduce the presence of embolic debris in the bloodstream. Such embolic filters are usually delivered in a collapsed position through the patient's vasculature and then expanded to trap the embolic debris. Some of these embolic filters are self expanding and utilize a restraining sheath which maintains the expandable filter in a collapsed position until it is ready to be expanded within the patient's vasculature. The physician can retract the proximal end of the restraining sheath to expose the expandable filter, causing the filter to expand at the desired location. Once the procedure is completed, the filter can be collapsed, and the filter (with the trapped embolic debris) can then be removed from the vessel. While a filter can be effective in capturing embolic material, the filter still needs to be collapsed and removed from the vessel. During this step, there is a possibility that trapped embolic debris can backflow through the inlet opening of the filter and enter the bloodstream as the filtering system is being collapsed and removed from the patient. Therefore, it is important that any captured embolic debris remain trapped within the filter so that particles are not released back into the body vessel.

Some prior art expandable filters are coupled to the distal end of a guide wire or guide wire-like member which allows the filtering device to be steered in the patient's vasculature as the guide wire is positioned by the physician. Once the guide wire is in proper position in the vasculature, the embolic filter can be deployed to capture embolic debris. The guide wire can then be used by the physician to deliver interventional devices, such as a balloon angioplasty dilatation catheter or a stent delivery catheter, to perform the interventional procedure in the area of treatment. After the procedure is completed, a recovery sheath can be delivered over the guide wire using over-the-wire techniques to collapse the expanded filter for removal from the patient's vasculature.

When a combination of an expandable filter and guide wire is utilized, it is important that the expandable filter portion should remain flexible in order to negotiate the often tortuous anatomy through which it is being delivered. An expandable filter which is too stiff could prevent the device from reaching the desired deployment position within the patient's vasculature. As a result, there is a need to increase the flexibility of the expandable filter without compromising its structural integrity once in position within the patient's body vessel. Also, while it is beneficial if the area of treatment is located in a substantially straight portion of the patient's vasculature, sometimes the area of treatment is at a curved portion of the body vessel which can be problematic to the physician when implanting the expandable filter. If the expandable filter portion is too stiff, it is possible that the filter may not fully deploy within the curved portion of the body vessel. As a result, gaps between the filter and vessel wall can be formed which may permit some embolic debris to pass therethrough. Therefore, the filtering device should be sufficiently flexible to be deployed in, and to conform to, a tortuous section of the patient's vasculature, when needed.

Embolic filtering devices typically include a filter element coupled to a self-expanding frame. The frame of the device must serve several purposes, such as providing the radial force to deploy the filter element from its delivered state, holding the filter element open in apposition to the wall of the body vessel, and aid in recovering the filter without spilling its contents. The frame must function in two states, collapsed and deployed. In the collapsed state, the frame must have the smallest profile possible. In cycling from the collapsed state to the deployed state, certain segments of the frame may require a large range of motion without permanent deformation. This has typically been achieved by utilizing superelastic materials, such as nickel-titanium, commonly known as nitinol.

A bending process is often used when making the frame from nitinol. Mechanical bending is an involved process with costly and complex tooling. Additionally, the process may involve multiple steps if forming a complex frame shape. Furthermore, with mechanical bending it is difficult to produce a consistent shape unless the starting material is completely straight. If there is a cast or helix in the wire or tube, it translates into variation in the final shape of the part.

Conventional guide wires for angioplasty and other vascular procedures usually comprise an elongated core member with one or more tapered sections near the distal end and a flexible body such as a helical coil disposed about the distal section of a core member. A shapeable member, which may be the distal extremity of the core member or a separate shaping ribbon that is secured to the distal extremity of the core member, extends through the flexible body and is secured to a rounded plug at the distal end of the flexible body. A torque applying mechanism is provided on the proximal end of the core member to rotate, and thereby steer, the guide wire while it is being advanced through the patient's vascular system.

A major requirement for guide wires and other guiding members, whether they are solid wire or tubular members, is that they have sufficient column strength to be pushed through a patient's vascular system or other body lumen without kinking. However, they must also be flexible enough to avoid damaging the blood vessel or other body lumen through which they are advanced. Efforts have been made to improve both the strength and flexibility of guide wires to make them more suitable for their intended uses, but these two properties are for the most part diametrically opposed to one another in that an increase in one usually involves a decrease in the other.

The prior art makes reference to the use of alloys, such as nitinol (NiTi alloy) and Beta titanium, which have shape memory and/or superelastic characteristics for medical devices that are designed to be inserted into a patient's body. Because of these properties, nitinol and Beta titanium have been employed in the fabrication of guide wires.

What has been needed is an expandable filter assembly having high flexibility with sufficient strength to be successfully deployed within a patient's vasculature to collect embolic debris which may be released into the patient's vasculature. What has also been needed is an expandable filter assembly having a frame made with reduced manufacturing costs resulting from lowered by a reduction in processing steps, tooling, operators and time. Further, there is a need to eliminate the problem of part variability that occurs due to cast and/or helix in the material of which the frame is made. The present invention disclosed herein satisfies these and other needs. The present invention can further be applied to guide wires.

### SUMMARY OF THE INVENTION

The present invention provides a flexible self-expanding frame for use with an embolic filtering device designed to capture embolic debris created during the performance of a therapeutic interventional procedure, such as a balloon angioplasty or stenting procedure, in a body vessel. The present invention provides the physician with an embolic filtering device having the flexibility needed to be steered through tortuous anatomy, but yet possesses sufficient strength to hold open a filtering element against the wall of the body vessel for capturing embolic debris. An embolic filtering device made in accordance with the present invention is relatively easy to deploy and has good flexibility and conformability to the patient's anatomy.

An embolic filter assembly of the present invention utilizes an expandable frame made from a self-expanding material, for example, a Beta titanium alloy, such as a Beta III titanium alloy, and includes a number of outwardly extending struts capable of expanding from an unexpanded position having a first delivery diameter to an expanded or deployed position having a second implanted diameter. In another embodiment of the present invention, the frame is made from a pair of half frames capable of expanding from an unexpanded position having a first delivery diameter to an expanded or deployed position having a second expanded diameter. A filter element made from an embolic-capturing material is coupled to the expandable frame to move between the unexpanded position and deployed position.

The struts of the frame can be set to remain in the expanded, deployed position until an external force is placed over the struts to collapse and move the struts to the unexpanded position. One way of accomplishing this is through the use of a restraining sheath, for example, which can be placed over the filtering device in a coaxial fashion to contact the frame and move the frame into the unexpanded position. The embolic filtering device can be implanted in the patient's vasculature and remain implanted for a period of time or can be coupled to the distal end of an elongated member, such as a guide wire, for temporary placement in the vasculature. A guide wire may be used in conjunction with the filtering device when embolic debris is to be filtered during an interventional procedure. In this manner, the guide wire and filtering assembly, with the restraining sheath placed over the filter assembly, can be placed into the patient's vasculature. Once the physician properly manipulates the guide wire into the target area, the restraining sheath can be retracted to deploy the frame into the expanded position. This can be easily performed by the physician by simply retracting the proximal end of the restraining sheath (located outside of the patient). Once the restraining sheath is retracted, the self-expanding properties of the frame cause each strut to move in an outward, radial fashion away from the guide wire to contact the wall of the body vessel. As the struts expand radially, so does the filter element which will now be maintained in place to collect embolic debris that may be released into the bloodstream as the physician performs the interventional procedure. The guide wire is used by the physician to deliver the necessary interventional device into the area of treatment. The deployed filter element captures embolic debris created and released into the body vessel during the procedure. In other embodiments of the invention, the half frames which cooperatively form the expandable frame can be set to remain in the expanded, deployed position until the sheath is placed over the half frames to collapse and move the frames to the unexpanded position.

In one embodiment of the present invention, the frame includes a proximal strut assembly coupled to a distal strut assembly. A filtering element is coupled to the distal strut assembly and is expandable within the patient's vasculature for filtering purposes. The proximal strut assembly can be made from a short set of self-expanding struts and a self-expanding deployment ring which simultaneously expand to contact the wall of the body vessel once implanted therein. The distal strut assembly also can be made from self-expanding struts and a deployment ring. The frame may be made from a Beta titanium alloy, such as a Beta III titanium alloy.

In another embodiment of the present invention, the distal strut assembly may include only the expandable ring which is coupled to the filter to create a “wind sock” type of filter design that creates an extremely flexible and bendable distal portion. The expandable ring member can be made from self-expanding material and creates an inlet opening for the filtering element that maintains good wall apposition once implanted in the patient.

In another embodiment of the invention, the expandable frame includes a pair of half frames which cooperatively form the expandable frame of the embolic filter assembly. In one particular embodiment, each of the half frames includes a first control arm connected to a second control arm by a partial loop. The partial loop extends radially outward when placed in an expanded position so that a substantially circular loop is created by the two partial loops. The frame may be made from a Beta titanium alloy, such as a Beta III titanium alloy.

In another embodiment of the invention, the half frames can be mounted onto a filter support structure which allows the composite frame and filter element to rotate relative to the guide wire without twisting the filter. The filter support structure can be mounted between a pair of fittings located on the guide wire which limit or eliminate relative longitudinal movement between the filter assembly and the guide wire.

The use of Beta III titanium alloy for the frame provides a frame that experiences minimal, or no, permanent deformation when cycled between its collapsed and delivered state. The elastic modulus of Beta III titanium is significantly higher than, for example, pseudoelastic nitinol which is currently used for many embolic filtering device frames. The higher elastic modulus allows the frame to be made from smaller diameter tubing, thereby reducing the overall collapsed profile of the device.

The present invention is also directed to an elongated guide wire. The elongated guide wire comprises an elongated core having proximal and distal core sections, wherein the distal core section may include one material while the proximal core section includes a different material. An optional torque-transmitting tube joins the proximal and distal core sections together. At least one flexible body, such as a metallic helical coil, may be disposed about and secured to the distal core section. The guide wire core, including both the distal and proximal core sections, may optionally be made from one solid, uninterrupted section of material.

It is to be understood that the present invention is not limited by the embodiments described herein. The present invention can be used in arteries, veins, and other body vessels. Other features and advantages of the present invention will become more apparent from the following detailed description of the invention, when taken in conjunction with the accompanying exemplary drawings.

### BRIEF DESCRIPTION OF THE DRAWINGS

FIG. 1 is a perspective view of an embolic filtering device embodying features of the present invention.

FIG. 2 is an elevational view of the embolic filtering device of FIG. 1.

FIG. 3 is an elevational view, partially in cross section, of an embolic filtering device embodying features of the present invention as it is being delivered within a portion of a body vessel.

FIG. 4 is an elevational view, partially in cross section, similar to that shown in FIG. 3, wherein the embolic filtering device is deployed in its expanded, implanted position within the body vessel.

FIG. 5 is an elevational view of another embodiment of an embolic filtering device made in accordance with the present invention.

FIG. 6A is an elevational view, partially in cross-section, of the distal end of the embolic filtering device of FIG. 1.

FIG. 6B is an elevational view, partially in cross section, of the distal end of the embolic filtering device of FIG. 5.

FIG. 7 is an elevational view showing another particular embodiment of an embolic filtering device make in accordance with the present invention.

FIG. 8 is an elevational view showing another particular embodiment of an embolic filtering device make in accordance with the present invention.

FIG. 9 is an elevational view showing another particular embodiment of an embolic filtering device make in accordance with the present invention.

FIG. 10A is a perspective view of another embodiment of an embolic filtering device embodying features of the present invention.

FIG. 10B is a perspective view of the embolic filtering device of FIG. 10A shown without the filter element attached to the expandable frame.

FIG. 10C is a side elevational view of an embolic filtering system which includes the embolic filtering device of FIG. 10A and a delivery sheath.

FIG. 10D is a side elevational view of the proximal end of the embolic filtering device of FIG. 10A showing in greater detail the mounting of the pair of half frames to the filter coil.

FIG. 10E is a cross-sectional view taken along line 10E-10E from FIG. 10D.

FIG. 10F is an end view which shows the expanded half frames that form the expandable frame of the embolic filtering device of FIGS. 10A and 10B.

FIG. 10G is a side elevational view showing an offset positioning of half frames forming the expandable frame.

FIG. 10A is a side elevational view, partially in cross-section, of the embolic filtering system shown in FIG. 10C as it is being delivered within a body vessel.

FIG. 11B is a side elevational view, partially in cross section, similar to that shown in FIG. 11A, wherein the embolic filtering device is deployed in its expanded, implanted position within the body vessel.

FIG. 12 is a cross-sectional view showing a stranded wire encapsulated by a layer of polymeric material which can be used to form the frames of any of the embolic filtering devices made in accordance with the present invention.

FIG. 13A is a perspective view of another embodiment of an embolic filtering device embodying features of the present invention.

FIG. 13B is a perspective view of the embolic filtering device of FIG. 4B shown without the filter element attached to the expandable frame.

FIG. 13C is a side elevational view of the expandable frame shown in FIG. 4B.

FIG. 14A is a perspective view of another embodiment of an embolic filtering device embodying features of the present invention.

FIG. 14B is a perspective view of the embolic filtering device of FIG. 14A shown without the filter element attached to the expandable frame.

FIG. 14C is an enlarged view which shows the connection of the control arms and partial loop which form the expandable frame shown in FIG. 14B.

FIG. 15A is a side elevational view partially in section of a guide wire having features of the present invention.

FIG. 15B is a side elevational view partially in section of another embodiment of a guide wire embodying features of the present invention.

FIG. 15C is a side elevational view of another embodiment of a guide wire embodying features of the present invention.

FIG. 15D is a cross-sectional view taken along line 15D-15D of the guide wire shown in FIG. 15B.

FIG. 15E is a cross-sectional view taken along line 15E-15E of the guide wire shown in FIG. 15B.

### DETAILED DESCRIPTION OF THE PREFERRED EMBODIMENTS

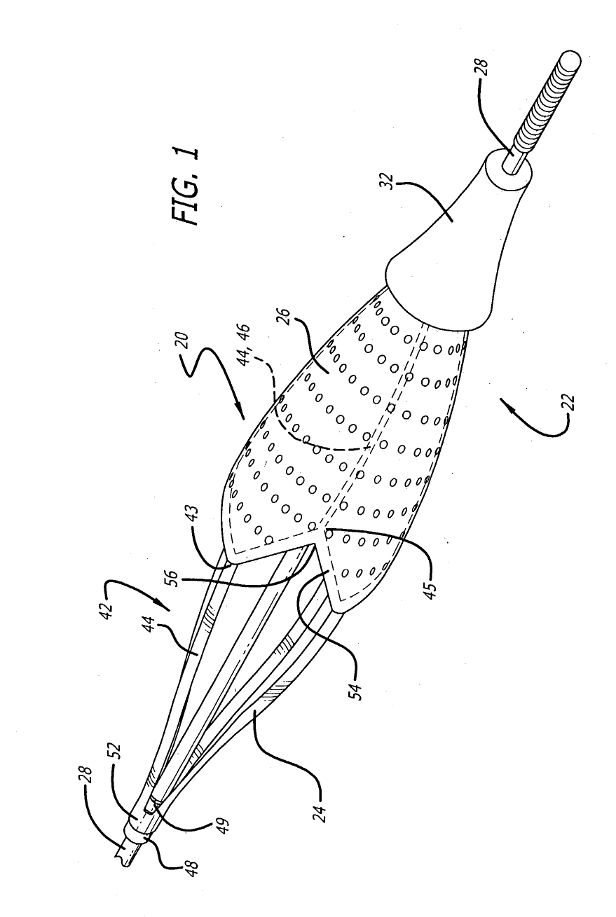

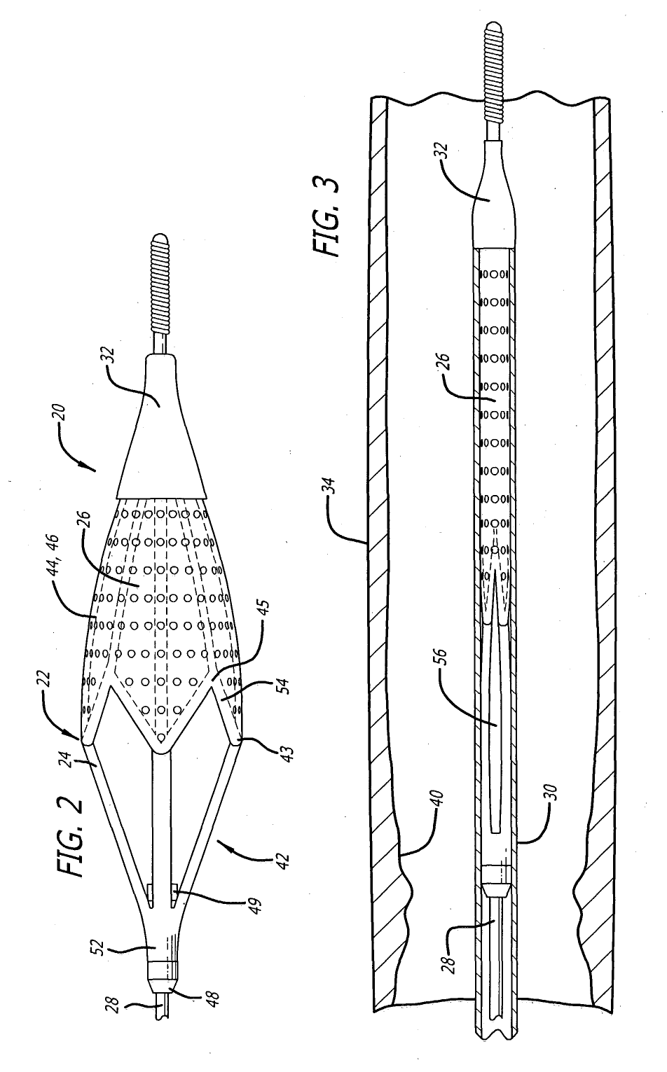

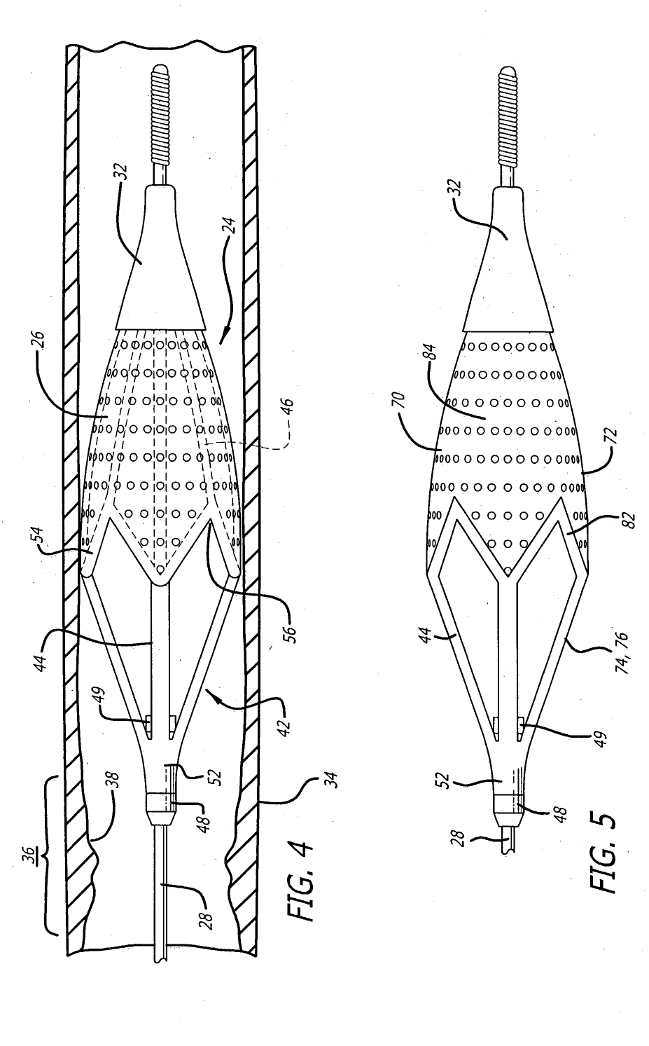

Turning now to the drawings, in which like reference numerals represent like or corresponding elements in the drawings, FIGS. 1 and 2 illustrate one particular embodiment of an embolic filtering device 20 incorporating features of the present invention. The embolic filtering device 20 is designed to capture embolic debris which may be created and released into a body vessel during an interventional procedure. The embolic filtering device 20 includes an expandable filter assembly 22 having a self-expanding frame 24 and a filter element 26 coupled thereto. In this particular embodiment, the expandable filter assembly 22 is rotatably mounted onto the distal end of an elongated tubular shaft, such as a steerable guide wire 28. A restraining or delivery sheath 30 (FIG. 3) extends coaxially along the guide wire 28 in order to maintain the expandable filter assembly 22 in its unexpanded position until it is ready to be deployed within the patient's vasculature. The expandable filter assembly 22 is deployed by the physician by simply retracting the restraining sheath 30 proximally to expose the expandable filter assembly. Once the restraining sheath is retracted, the self-expanding frame 24 becomes uncovered and immediately begins to expand within the body vessel (see FIG. 4), causing the filter element 26 to expand as well.

An optional obturator 32 affixed to the distal end of the filter assembly 22 can be implemented to prevent possible “snowplowing” of the embolic filtering device as it is being delivered through the vasculature. The obturator can be made from a soft polymeric material, such as Pebax D 40, and preferably has a smooth surface to help the embolic filtering device travel through the vasculature and cross lesions while preventing the distal end of the restraining sheath 30 from “digging” or “snowplowing” into the wall of the body vessel.

In FIG. 3, the embolic filtering device 20 is shown as it is being delivered within an artery 34 or other body vessel of the patient. In FIG. 4, the expandable filtering assembly 22 is shown in its expanded position within the patient's artery 34. This portion of the artery 34 has an area of treatment 36 in which atherosclerotic plaque 38 has built up against the inside wall 40 of the artery 34. The filter assembly 22 is placed distal to and downstream from the area of treatment 36. For example, the therapeutic interventional procedure may comprise the implantation of a stent (not shown) to increase the diameter of an occluded artery and increase the flow of blood therethrough. It should be appreciated that the embodiments of the embolic filtering device described herein are illustrated and described by way of example only and not by way of limitation. Also, while the present invention is described in detail as applied to an artery of the patient, those skilled in the art will appreciate that it can also be used in a variety of arteries or other body vessels, such as the coronary arteries, carotid arteries, renal arteries, saphenous vein grafts (SVG) and other peripheral arteries. Additionally, the present invention can be utilized when a physician performs any one of a number of interventional procedures, such as balloon angioplasty, laser angioplasty or atherectomy which generally require an embolic filtering device to capture embolic debris created during the procedure.

The frame 24 includes self-expanding struts which, upon release from the restraining sheath (not shown), expand the filter element 26 into its deployed position within the artery (FIG. 4). Embolic debris created during the interventional procedure and released into the bloodstream are captured within the deployed filter element 26. Although not shown, a balloon angioplasty catheter can be initially introduced within the patient's vasculature in a conventional SELDINGER technique through a guiding catheter (not shown). The guide wire 28 is disposed through the area of treatment and the dilatation catheter can be advanced over the guide wire 28 within the artery 34 until the balloon portion is directly in the area of treatment 36. The balloon of the dilatation catheter can be expanded, expanding the plaque 38 against the wall 40 of the artery 34 to expand the artery and reduce the blockage in the vessel at the position of the plaque. After the dilatation catheter is removed from the patient's vasculature, a stent (not shown) can be implanted in the area of treatment 36 using over-the-wire or rapid exchange techniques to help hold and maintain this portion of the artery 34 and help prevent restenosis from occurring in the area of treatment. The stent could be delivered to the area of treatment on a stent delivery catheter (not shown) which is advanced from the proximal end of the guide wire to the area of treatment. Any embolic debris created during the interventional procedure will be released into the bloodstream and will enter the filter 26. Once the procedure is completed, the interventional device may be removed from the guide wire. The filter assembly 22 can also be collapsed and removed from the artery 34, taking with it any embolic debris trapped within the filter element 26. A recovery sheath (not shown) can be delivered over the guide wire 28 to collapse the filter assembly 22 for removal from the patient's vasculature.

In one embodiment of the invention, the frame 24, shown in FIGS. 1-4, includes a proximal strut assembly 42 which includes a number of self-expanding struts 44 that extend radially outward from the unexpanded position, as shown in FIG. 3, to an expanded, implanted position as shown in FIG. 4. The proximal strut assembly 42 is coupled to a distal strut assembly 46 which also includes a number of self-expanding struts 44 that extend radially out once placed in the expanded position. The filter element 26 is coupled to the distal strut assembly 46 for filtering particles of emboli which may be released in the artery.

The proximal ends of the struts 44 of the proximal strut assembly 42 are coupled to a collar 52 which can be rotatably coupled to the guide wire 28. The distal ends of each strut 44 of the proximal strut assembly 42 are in turn coupled to a deployment ring 54, also made from a self-expanding material, which aids in the expansion of the proximal strut assembly 42. The deployment ring 54 is shown having a number of pleats 56 which helps when collapsing the ring 54 to its delivery profile, as shown in FIG. 3. The proximal ends of the struts 44 of the distal strut assembly 46 may likewise be coupled to the deployment ring 54. The deployment ring 54 is located at the opening of the filter element 26 to help provide proper wall apposition when placed in the body vessel. In this regard, the deployment ring 54 helps to insure that the filter element 26 is properly placed against the vessel wall 40 to prevent the formation of gaps which might otherwise form between the filter and the vessel wall. The pleats 56 of the deployment ring 54 also help to prevent the filter 26 from entering a recovery sheath (not shown) when the filter assembly 22 is to be collapsed for removal from the patient. The deployment ring 54 is shown having a zigzag pattern which forms peaks 43 and valleys 45, but may include other patterns, such as undulations. As a result, the filter 26 and frame 24 will enter the recovery sheath in a smooth fashion, which may help to prevent collected emboli from back washing into the body vessel.

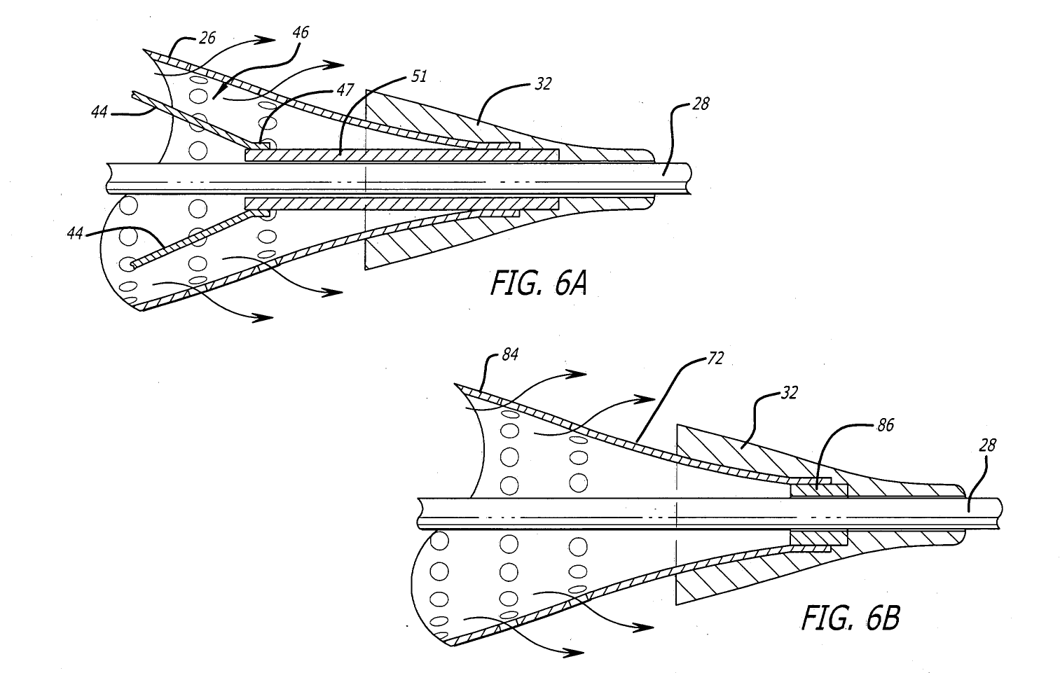

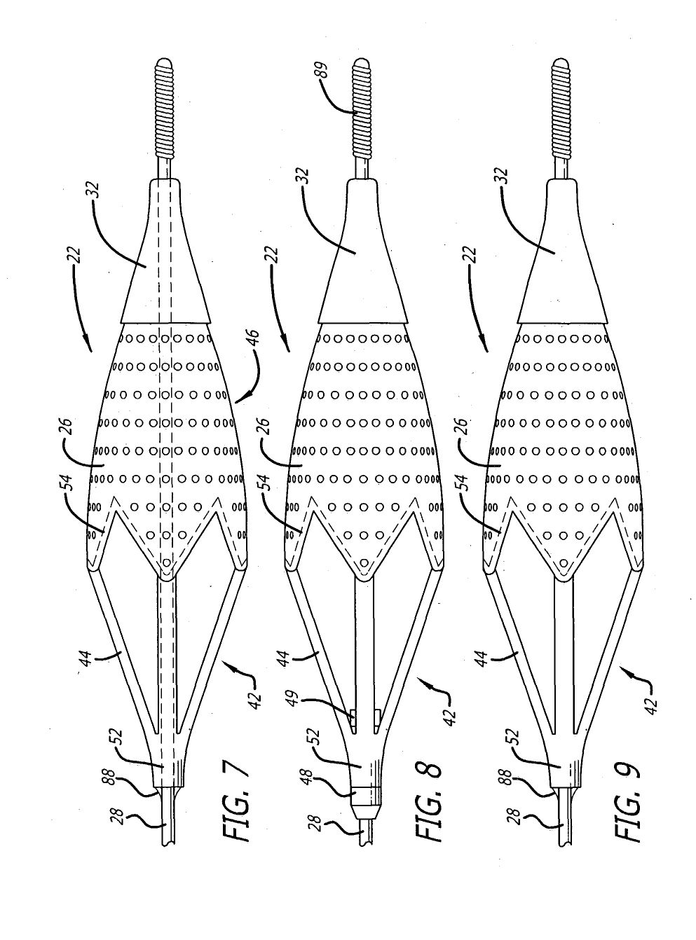

Referring particularly to FIG. 6A, a collar 47 may be coupled to the distal ends of the struts 44 of the distal strut assembly 46. The collar 47 can be coupled to a tubular member 51 which is placed over the guide wire 28 to allow the distal strut assembly 46 to rotate on the guide wire 28 and permit the assembly to move in a longitudinal direction along the guide wire as it moves between the unexpanded position and the expanded position. The tubular member 51 can be made from a polymeric material and would be bonded or otherwise coupled to the distal end of the filter 26 as well. The obturator 32 could also be adhesively bonded or otherwise coupled to the tubular member 51. Thus, the obturator 32 would then be rotatable and slidable along the guide wire 28 as well. A pair of stop fittings 48 and 49 (see FIG. 2) coupled to the guide wire 28 maintains the collar 52 of the proximal strut assembly 42 in place and inhibits or limits longitudinal movement of the proximal strut assembly 42 along the guide wire. Thus, the frame 24 will spin or rotate about the guide wire 28. It should be appreciated that in an alternative design, the collar of the distal strut assembly 46 could be fixed to the guide wire allowing the proximal strut assembly to move longitudinally along the guide wire to allow the frame 24 to expand and collapse. Still other configurations can be implemented for coupling the filter assembly 22 to the guide wire 28, such as those shown in FIGS. 7-9.

Referring now to FIGS. 5 and 6B, an alternative embodiment of an embolic filtering device 70 is shown. This particular embodiment of the embolic filtering device 70, sometimes referred to as a “wind sock” design, is similar to the previously described filter device 20 of FIGS. 1 and 2. The filtering assembly 72 includes a frame 74 having only a proximal strut assembly 76 and a deployment ring 82 which is coupled to a filtering element 84. This particular embodiment functions in the same manner as the embodiment of FIGS. 1 and 2 described above.

The embolic filtering device 70 shown in FIG. 5 can be rotatably mounted to the guide wire 28 as is shown in FIG. 5. A pair of stop fittings 48 and 49 can be utilized to fix the proximal strut assembly 76 to the guide wire 28. As can be seen in FIG. 6B, the distal-most end of the filtering assembly 72 is rotatably mounted onto the guide wire 28. To achieve rotatability, the distal end of the filtering element 84 can be affixed to a rotatable collar 86 coupled onto the obturator 32. An optional obturator 32 encases the distal end of the filtering element 84 to the guide wire. It should be appreciated that the obturator 32 can also be rotatably mounted onto the guide wire 28 to allow the filtering assembly to spin freely on the guide wire.

Referring now to FIGS. 7-9, alternative methods for mounting the embolic filtering assembly 22 to the guide wire 28 are shown. Referring initially to FIG. 7, the embolic filtering assembly 22 is shown with the collar 52 affixed to the guide wire 28 to prevent any rotating or spinning of the filtering assembly 22. As can be seen in FIG. 7, a weld 88 can be used to permanently secure the proximal assembly 42 to the guide wire 28. The distal strut assembly 46 making up the filter assembly 22 can be similar to the distal strut assembly shown in FIG. 1, and can include a set of struts that can be coupled to the guide wire 28 in a similar fashion as is shown in FIG. 6A. Alternatively, the filtering assembly could be made with the “windsock” design shown in FIGS. 5 and 6B.

Referring now to FIG. 8, the embolic filtering assembly 22 is shown in an alternative form as it is mounted onto an elongated member, such as a guide wire 28. In this particular embodiment, it should be noted that the guide wire 28 terminates at the location of the stop fitting 49 and does not extend through the embolic filtering assembly 22, as does the guide wire 28 shown in FIG. 7. In this manner, the embolic filtering assembly 22 can be collapsed to a small profile in the unexpanded position which may be beneficial when attempting to implant the device in a small diameter body vessel. As can be seen in FIG. 8, the collar 52 of the proximal strut assembly 42 is still rotatably mounted onto the distal end of the guide wire 28 by a pair of stop fittings 48 and 49. The distal end of the filter assembly 22 may include a coil tip 89 which could be utilized to maneuver the device through the patient's vasculature. In this manner, a short section of wire which includes the coil tip 89 could be bonded, for example, to the tubular member 51 shown in FIG. 6A. Adhesives or similar bonding-techniques could be utilized to couple the coil tip to the tubular member 51. FIG. 9 shows another embodiment of the embolic filtering assembly 22 as it is affixed to the guide wire 28. This particular embolic filtering assembly 22 is similar to that shown in FIG. 8 in that the guide wire 28 terminates at the collar 52 of the proximal strut assembly 42. It is similar to the assembly shown in FIG. 7 in that the collar 52 is secured to the guide wire 28 using welding or other coupling means to maintain the collar 52 permanently affixed to the distal end of the guide wire 28. In this manner, the embolic filtering assembly of FIG. 9 should not spin freely on the guide wire. However, as with the embodiment shown in FIG. 8, the guide wire does not extend through the filtering assembly in order to create a small profile when placed in the unexpanded position. It should be appreciated that both the embolic filtering assemblies of FIGS. 8 and 9 may include a distal strut assembly that may include struts, such as shown in FIGS. 1 and 6A, or can be of the “windsock” design shown in FIGS. 5 and 6B.

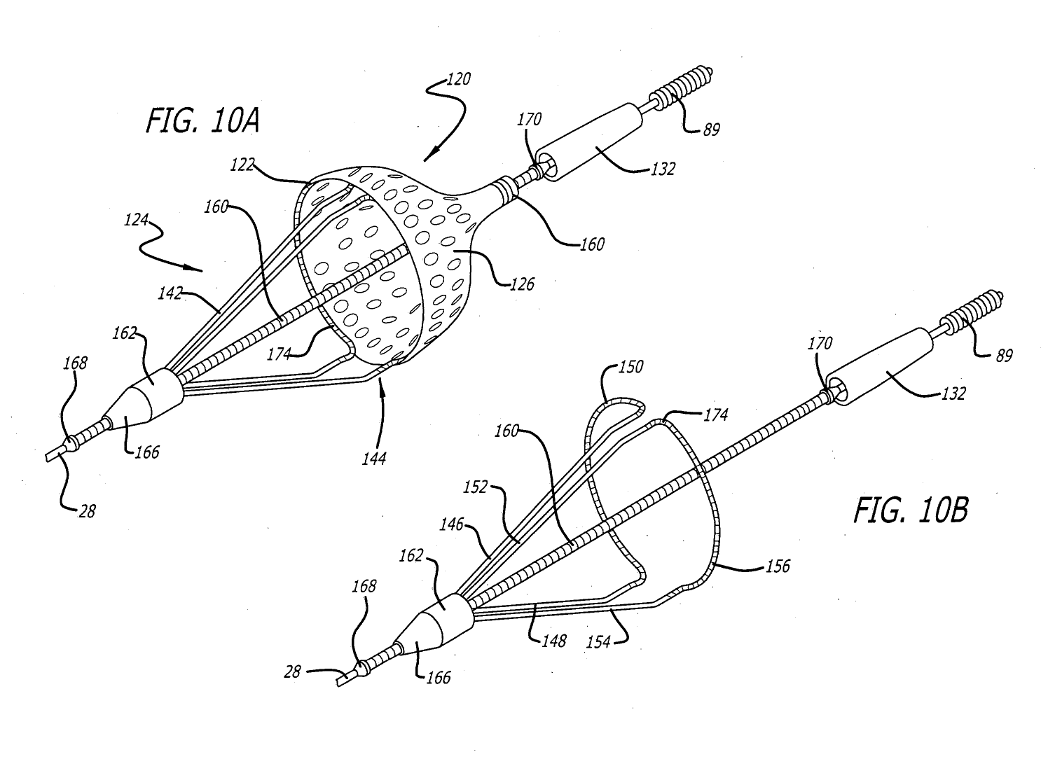

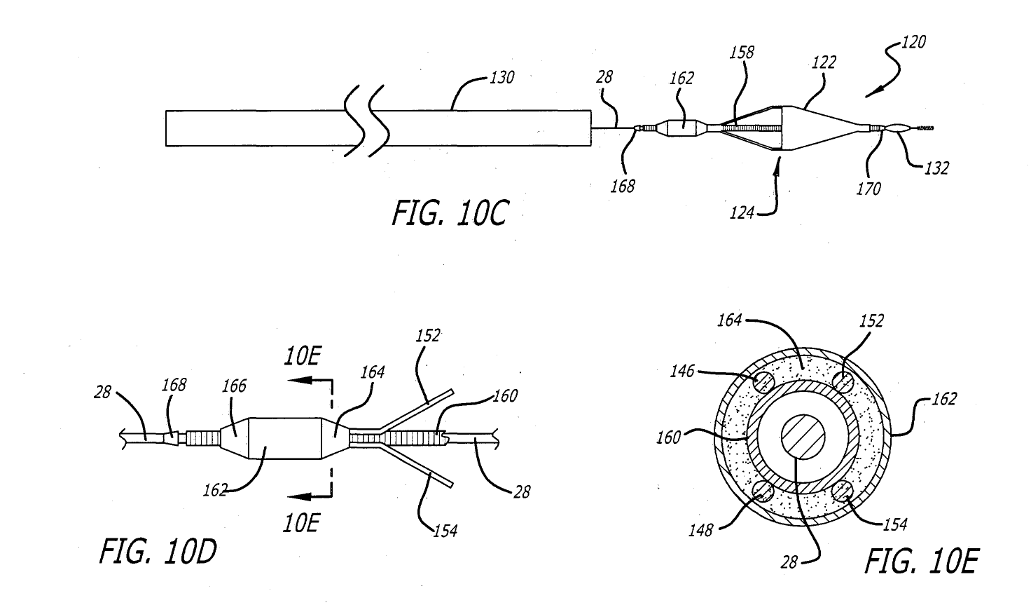

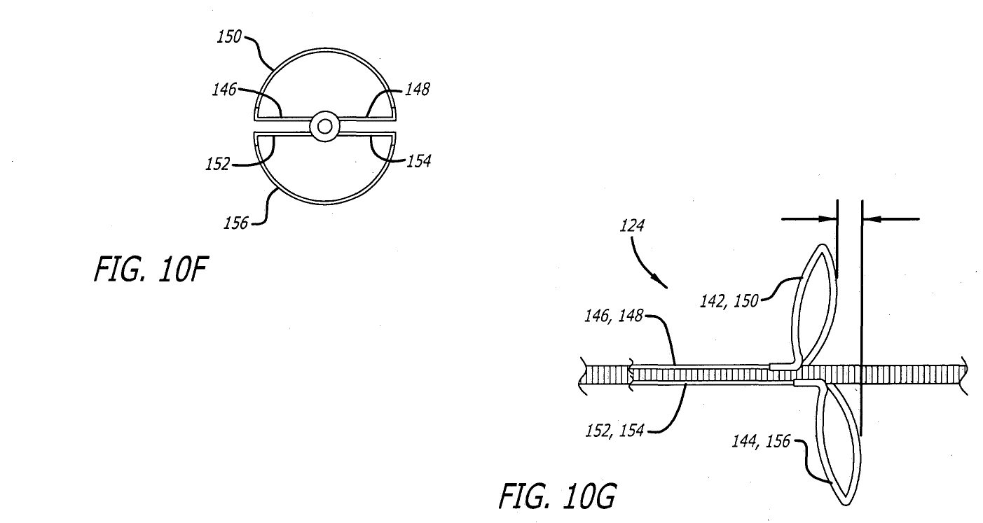

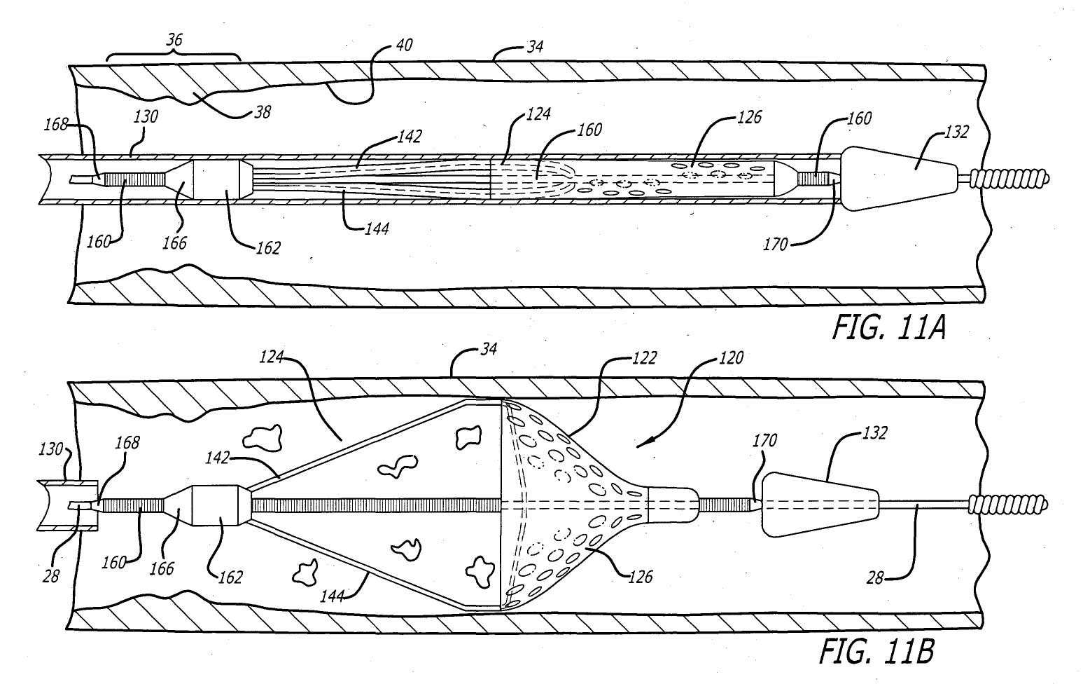

Turning now to the drawings, in which like reference numerals represent like or corresponding elements in the drawings, FIGS. 10A, 10B and 10C illustrate another embodiment of an embolic filtering device 120 incorporating features of the present invention. An expandable filter assembly 122 is rotatably mounted near the distal end of a steerable guide wire 28. A restraining or delivery sheath 130 (see FIGS. 10C and 11A) extends coaxially along the guide wire 28 in order to maintain the expandable filter assembly 122 in its unexpanded, delivery position until it is ready to be deployed within the patient's vasculature. An optional obturator 132 is affixed to the guide wire 28 distal to the filter assembly 122.

In FIGS. 11A and 11B, the embolic filtering device 120 is shown as it is being delivered within the artery 34 or other body vessel of the patient. Referring specifically to FIG. 11B, the embolic filtering assembly 122 is shown in its expanded position within the patient's artery 34. The expandable frame 124 includes a pair of half frames 142 and 144 (also referred to as D-frames) which, upon release from the restraining sheath 30, expand the filter element 126 into its deployed position within the artery (FIG. 11B). Embolic debris created during the interventional procedure and released into the body fluid are captured within the deployed filter element 126.

Referring specifically to FIGS. 10A-10F, the particular embodiment of the frame 124 includes a first half frame 142 and second half frame 144 which cooperatively form a deployment mechanism for expanding the filter element 126 within the patient's vasculature. As can be seen in these figures, the first half frame 142 includes a first control arm 146 and a second control arm 148 connected to each other via a partial loop 150 which extends radially outward once placed in the deployed position as is shown in FIG. 10B. Likewise, the second half frame 144 includes a first control arm 152 and a second control arm 154 connected by a partial loop 156. These partial loops form a D-shaped structure when placed in an expanded position. Once placed in the deployed position, as is shown in FIG. 10B, the partial loops 150 and 156 cooperatively form a composite circular shaped loop having a large opening to which the filter element 126 is coupled. In this fashion, once the first half frame 142 and the second half frame 144 are deployed, the partial loops 150 and 156 will self-expand radially to contact the wall of the artery to maintain proper wall apposition to prevent gaps from forming between the filter element 126 and the wall of the body vessel. These half frames are sometimes referred to as D-frames since the partial loops form a D-shape once deployed (see FIG. 10F). Any embolic debris or unwanted particles which may be entrained in the body fluid passing through the body vessel should be captured in the filter element.

The filtering assembly 122 is rotatably mounted onto the guide wire 28 via a filter support structure 158. The filter support structure 158, shown in the embodiment of FIGS. 10A-10F as a filter coil 160, provides a suitable amount of flexibility and bendability to the composite filter assembly as the device is being delivered through the sometimes tortuous paths leading to the area of treatment. As can be seen in FIGS. 10A and 10B, the filter coil 160 can extend from a position proximal to the frame 124 to a position distal to the end of the filter element 126. While a wire coil is utilized to form the filter coil 160, it should be appreciated by those skilled in the art that other components could be utilized to create the filter support structure 158 without departing from the spirit and scope of the present invention. For example, a piece of tubing having good flexibility could also be utilized as the filter support structure. One suitable material for the filter coil includes 304 stainless steel spring wire having a diameter of about 0.051±0.005 mm (0.002±0.0002 inches).

As can best be seen in FIGS. 10A-10C, each of the first and second control arms of the first half frame 142 and the second half frame 144 are connected at a sleeve or collar 162 located proximal to the partial loops 150 and 156. In this regard, the ends of each of the first and second control arms are connected substantially together by the collar 162. The collar 162 can be mounted over the ends of the first and second half frames to maintain the ends fixedly disposed between the collar 162 and the filter coil 160. The collar 162 can be made from a highly radiopaque material such as a platinum/iridium alloy having a material composition of 90% platinum and 10% iridium. More specifically, FIGS. 10D and 10E show one particular arrangement for mounting the half frames to the filter coil 120. Solder 164 is placed over the ends of the first and second half frames in order to create a smooth, tapered surface with the outer surface of the collar 162. A tapered solder joint 166 located proximal to the collar 162 also can be utilized to help maintain the first and second half frames mounted onto the filter coil 160. The solder joint 166 also provides a smooth taper with the outer surface of the collar 162. It will be appreciated by those skilled in the art that still other ways of mounting the first and second half frames onto the filter support structure 158 can be implemented in accordance with the present invention.

As can best be seen in FIGS. 10A-10C, the filter assembly 122 is disposed between a proximal stop fitting 168 and distal stop fitting 170 placed on the guide wire 28. In this manner, the stop fittings 168 and 170 abut against the ends of the filter coil 160 to either inhibit longitudinal motion of the filter assembly 122 relative to the guide wire completely or to provide a limited range of motion along the guide wire. As is shown in the same figures, the proximal fitting 168 and distal fitting 170 are placed in close proximity to the ends of the filter coil 160 to prevent any appreciable amount of longitudinal motion of the filter assembly 122 relative to the guide wire 28. However, the spacing between the proximal fitting 168 and distal fitting 170 could be increased to allow a limited range of motion of the filter assembly relative to the guide wire. Additionally, this particular mounting system allows the filter assembly 122 to be rotatably mounted onto the guide wire 28 to permit the guide wire to rotate freely once the first and second half frames 142 and 144 are deployed in the body vessel. In this manner, if the physician should spin the guide wire at its proximal end while placing an interventional device on the guide wire, that rotation will not be transmitted to the deployed wire frame 124. Thus, the frame 124 and the filter element 126 should remain stationary in the event of accidental or intentional rotation of the guide wire at its proximal end.

Referring now to FIG. 11A, the first half frame 142 and second half frame 144 are shown in a collapsed, delivery position within the restraining sheath 130. As can be seen in FIG. 11A, the first and second control arms and partial loop forming the half frames actually define a single, complete loop which extends in a longitudinal fashion within the restraining sheath 130. Once the restraining sheath 130 has been retracted, the self-expanding properties of the material used to manufacture the first and second half frames 142 and 144 allow the partial loops to expand radially outward to the deployed position shown in FIG. 11B. The control arms will expand radially outward to some degree as well. Once deployed, the partial loops 150 and 156 cooperatively form a complete circular loop which forms an opening for the filter element 126.

Referring now to FIG. 10G, a particular embodiment of an expandable frame 124 is shown with the lengths of the first and second control arms of the first and second half frames 142 and 144 being varied to achieve an offset or gap between the two half frames. As can best be seen particularly in FIG. 10G, the first and second control arms 146 and 148 of the first half frame 142 have a length which is longer than the length of the first and second control arms 152 and 154 of the second half frame 144. The length of the control arms is generally measured from the end of the arm as mounted to the collar 162 up to the transition area where the partial loop starts to extend radially away from the arm in the deployed position. In this manner, the first half frame 142 has control arms of unequal length to the second half frame 144 which may be useful when deploying the filtering assembly 122 in curved portions of the anatomy. As a result, when the frame 124 is expanded into its deployed expanded position, the differences in the lengths of the control arms create a gap between the positioning of the partial loops 150 and 156. The gap is indicated by arrows in FIG. 10G. Additionally, the offset or gap between the first and second half frames helps when retracting the filter assembly back into a recovery sheath or into the delivery sheath since the length of the first and second half frames will be different in the collapsed delivery position as well. As a result, one of the half frames will take a position that is not in direct contact with a portion of the other half frame which may make it easier to retrieve the frame into either the delivery or recovery sheath.

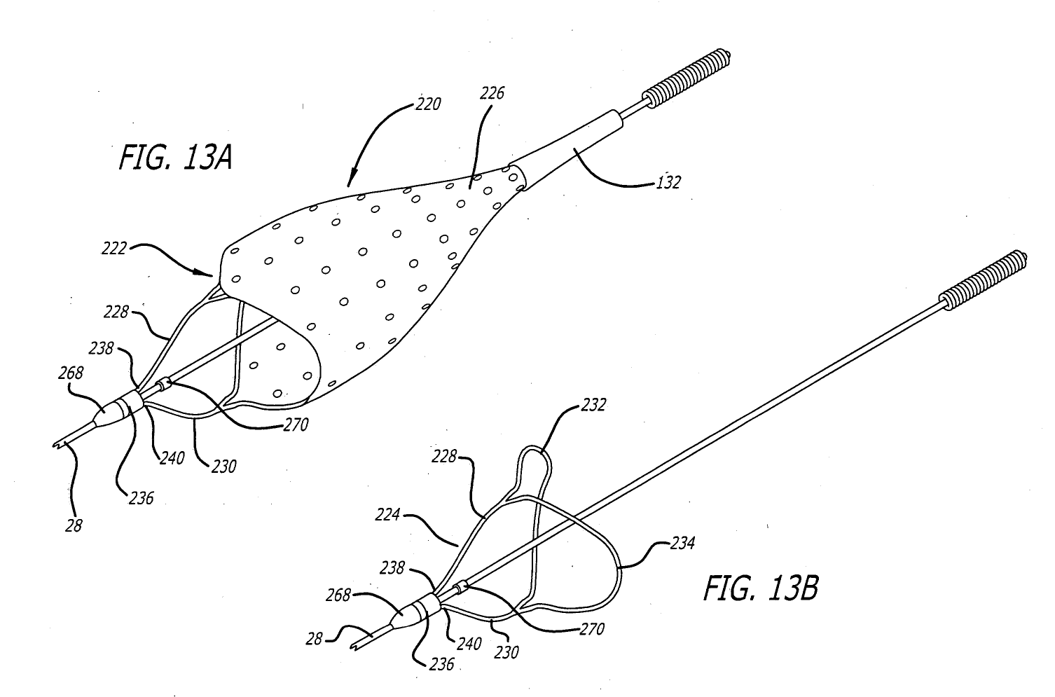

Referring now to FIGS. 13A-13C, an alternative embodiment of an embolic filtering device 220 is shown. This particular variation includes a filter assembly 222 having an expandable frame 224 to which is coupled a filtering element 226. The expandable frame 224 is different from the other disclosed embodiments of the expandable frame in that a single frame is utilized, rather than two separate half frames which cooperatively form the expandable frame. In this regard, the expandable frame 224 includes a pair of control arms 228 and 230 which extend from a proximally mounted location to a divergence where a pair of partial loops 232 and 234 is connected. The partial loops 232 and 234 function in substantially the same manner as the previously described loops in order to expand together to form a substantially circular diameter used as a deployment means for maintaining the filter element 226 deployed within the body vessel. The ends 233 and 235 of these control arms 228 and 230 translate to a substantially Y-shaped transition region where the partial loops 232 and 234 of the frame are connected. In this particular embodiment, the expandable frame 224 eliminates a set of control arms by creating a single set of arms which can be expanded while holding the filtering element 226 in place utilizing a pair of partial loops 232 and 234. This particular embodiment utilizes a collar 236 to which the proximal ends 238 and 240 of the control arms are coupled. This particular collar 236 can be rotatably mounted onto the guide wire 28 to permit rotation between components. Additionally, the proximal fitting 268 and distal fitting 270 can be placed onto the guide wire to limit or eliminate relative longitudinal motion between the filtering assembly 222 and the guide wire 28. Although not shown in FIGS. 13A-13C, this particular embodiment also could be mounted onto a filter support structure, such as a filter coil 60, as previously discussed.

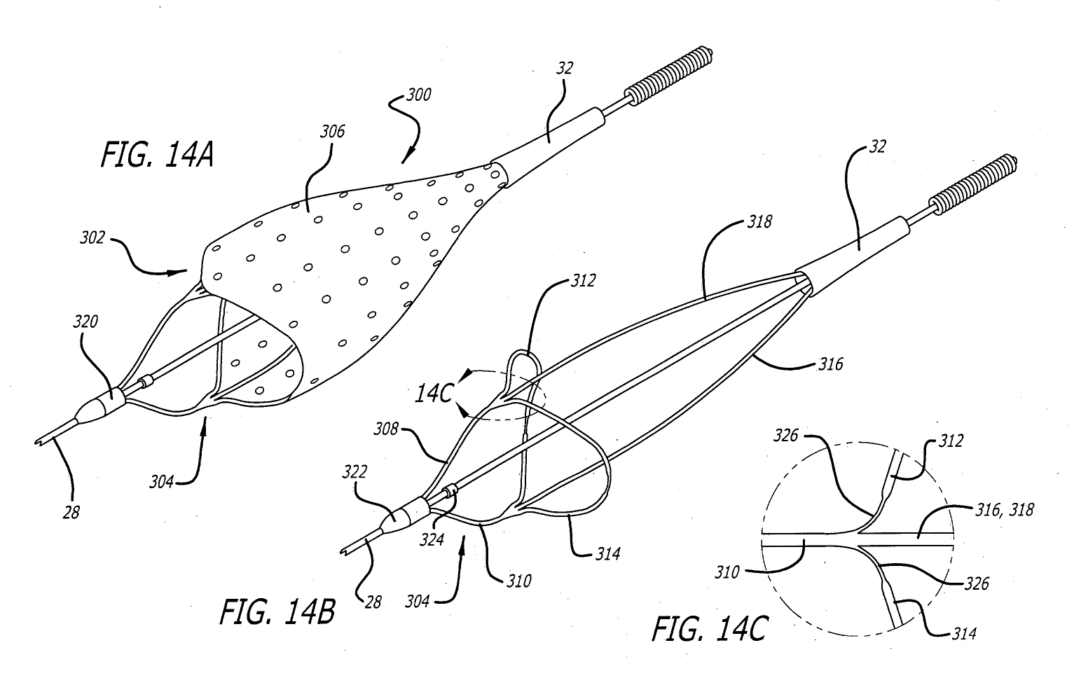

Referring now to FIGS. 14A-14C, yet another embodiment of a filtering device 300 is shown. This particular embodiment of an embolic filtering device 300 includes a filter assembly 302 having an expandable frame 304 which is best seen in FIG. 14B. A filter element 306 is coupled to the expandable frame 304 in order to collect unwanted particles which may be entrained in the body fluid of a body vessel. The filter assembly 302 is mounted to a guide wire 28 similarly to the previously described embodiments. This particular expandable frame 304 is similar to the embodiment of FIGS. 13A-13C, in that the expandable frame includes a pair of flexible control arms 308 and 310 from which extends a pair of partial loops 312 and 314. The expandable frame 304, however, includes an additional set of distal control arms 316 and 318 which extend distally from the connection point where the proximal control arms 308 and 310 are connected to the partial loops 312 and 314. The pair of distal control arms 316 and 318 extend distally to a collar (not shown) which is rotatably mounted onto the guide wire. Likewise, the ends of the proximal control arms 308 and 310 are coupled to a rotatable collar 320 which allows the expandable frame 304 to spin relative to the guide wire. A proximal stop fitting 322 and a distal stop fitting 324 are placed on the guide wire to prevent or limit the amount of longitudinal movement of the filter assembly 302 relative to the guide wire.

Referring to FIG. 14C, the “Y” shaped connection of the expandable frame 304 is shown in greater detail. As can be seen in this particular figure, the ends of the partial loops have strain distributing struts 326, shown as thin strut widths that enhance bendability at bend points in the frame. The distal control arm 316 which extends from the “Y” junction can have the same strut width as the proximal control arm 308, as shown in FIG. 14C, or it may include a thinner strut to allow the distal control arms to bend more freely. Likewise, the proximal control arms may also have a smaller strut width proximal to the “Y” transition to allow the frame to bend more easily and to help reduce and distribute the strain developed when the frame moves between collapsed and expanded positions.

The expandable frames of the present invention can be made in many ways. One particular method of making the frames 24 (FIGS. 1 and 2) and 74 (FIG. 5) is to cut a thin-walled tubular member, such as a hypotube, to remove portions of the tubing in the desired pattern for each strut, leaving relatively untouched the portions of the tubing which are to form each strut. Similarly, a tubular member, such as a hypotube, may be cut to remove portions of the tubing in the desired pattern for each half frame or full frame of the expandable frames 124 (FIGS. 11A-11B), 224 (FIGS. 13A-13C) and 304 (FIGS. 14A-14C), leaving relatively untouched the portions of the tubing which are to form the control arms and partial loop(s). The tubing may be cut into the desired pattern by means of a machine-controlled laser. Prior to laser cutting the strut pattern, the tubular member could be formed with varying wall thicknesses which may be used to create flexing portions within the frame. Alternatively, the expandable frames 124, 224 and 304 may be made from a wire possessing self-expanding properties.

The thin-walled tubular members, or hypotubes, may include Beta titanium or Beta III titanium. Titanium alloys are generally classified as alpha, beta, or mixed alpha-beta depending on the metallurgical stability of the alloy's crystalline phases at room temperature. Under equilibrium conditions, pure titanium has an alpha structure up to about 880° C. (1620° F.), above which it transforms to a beta structure. The inherent properties of alpha and beta structures are quite different. Through alloying and heat treatment, one or the other or a combination of these two structures can be made to exist at service temperatures, such as at room temperature, and the properties of the material vary accordingly.

Although there is no concise definition for beta titanium alloys, near-beta alloys and metastable-beta alloys are often referred to as classes of beta titanium alloys. Near-beta alloys are generally alloyed with appreciably higher beta stabilizers than conventional alpha-beta alloys, but are not sufficiently stabilized to readily retain an all-beta structure. Near-beta phase alloys are typically treated below the beta transus to produce primary alpha phase which in turn results in an enriched, more stable beta phase.

Metastable-beta phase titanium alloys are even more heavily alloyed with beta stabilizers than near-beta alloys and readily retain an all-beta structure. The added stability of these alloys eliminates the need to heat treat below the beta transus to enrich the beta phase. The term “metastable” is used for these alloys because the beta phase in them is not truly stable. In fact, these alloys can be aged to advance the alpha phase to strengthen the material.

Three representative beta titanium alloys include: Ti-15V-3Cr-35n-3Al; Ti-13V-11Cr-3Al; and Ti-3Al-8V-6Cr-4Mo-4Zr. Beta III titanium was developed to supplement the Ti-13V-11Cr-3Al class of Beta-titanium alloys. Beta III titanium (Ti-11.5Mo-6Zr-4.5Sn) is a metastable-beta phase titanium alloy including titan(ium with about 11.5 wt % molybdenum, 6 wt %, zirconium, and 4.5 wt % tin. In various Beta III titanium compositions, the quantity of molybdenum can range from about 10-13 wt %, the zirconium can range from about 4.5-7.5 wt %, and the tin can range from about 3.75-5.25 wt %.

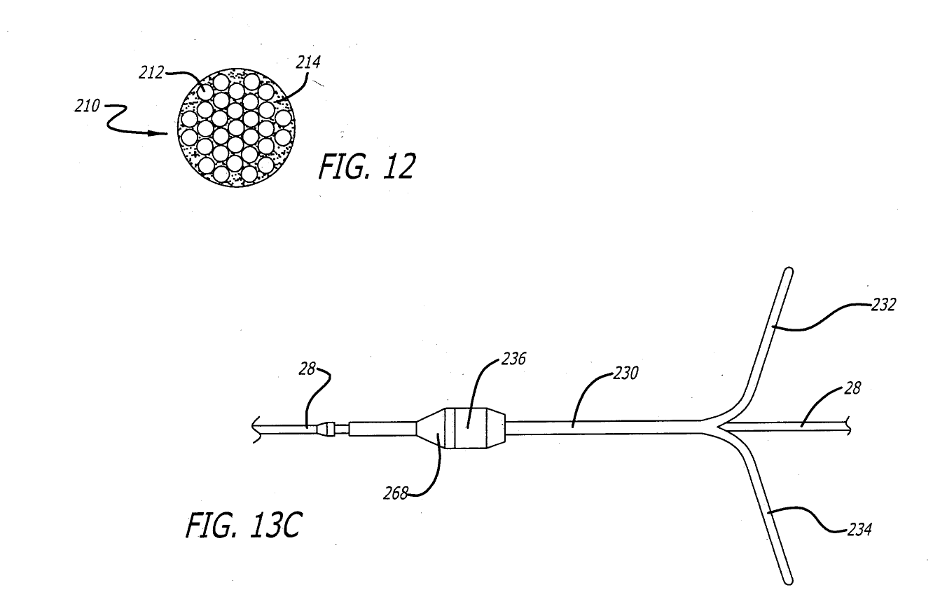

Referring now to FIG. 12, a cross-sectional view of one type of wire which can be utilized in creating the expandable frames 124, 224 and 304 is shown. In FIG. 12, a composite wire 210 is made from a number of wire strands 212 which cooperate to form a single wire. These multiple strands 212 forming the composite wire can be encapsulated by a polymeric material 214, such as polyurethane, to help prevent the strands 212 from unraveling during assembly or use. Alternatively, the expandable frame could be formed by a single wire, rather than multiple wire strands. It should be appreciated that any of the embodiments of the invention described herein could be made from either a single solid wire or multiple wire strands without departing from the spirit and scope of the present invention. When a multiple strand wire is utilized to create the frame, it is possible that some of the wire strands could be made from different materials other than, for example, Beta titanium, or more particularly, Beta III titanium. In this regard, some of the strands could be made from a material having higher radiopacity than Beta titanium to enhance the visualization of the expandable frame during fluoroscopy. Radiopaque coils (not shown) could be wrapped around the partial loops of the half frames to increase visualization during fluoroscopy.

The expandable frames are often very small, so the tubing or wire from which the frame is made must necessarily have a small diameter. Typically, the tubing has an outer diameter of about 0.51-1.02 mm (0.020-0.040 inches) in the unexpanded condition. The wall thickness of the tubing is usually about 0.08-0.15 mm (0.003-0.006 inches). The diameter of the wire that can be used to form the frames 124, 224 and 304 can be as small as about 0.091 mm (0.0036 inches). Of course, large diameter wire could be used as well. When multiple stranded wire is utilized, the diameter of the composite wire can be about 0.15 mm (0.006 inches). As can be appreciated, the width and/or thickness at the strain distributing strut or bending points will be less. For frames implanted in body lumens, such as PTA applications, the dimensions of the tubing may be correspondingly larger. While it is preferred that the frame be made from laser cut tubing, those skilled in the art will realize that the frame can be laser cut from a flat sheet and then rolled up in a cylindrical configuration with the longitudinal edges welded to form a cylindrical member.

Generally, when the frame or a half frame is to be cut, the tubing is put into a rotatable collet fixture of a machine-controlled apparatus for positioning the tubing relative to a laser. According to machine-encoded instructions, the tubing is then rotated and moved longitudinally relative to the laser which is also machine-controlled. The laser selectively removes the material from the tubing by ablation and a pattern is cut into the tube. The tube is therefore cut into the discrete pattern of the finished frame. The frame can be laser cut much like a stent is laser cut. Details on how the tubing can be cut by a laser are found in U.S. Pat. No. 5,759,192 (Saunders), U.S. Pat. No. 5,780,807 (Saunders) and U.S. Pat. No. 6,131,266 (Saunders) which have been assigned to Advanced Cardiovascular Systems, Inc.

The process of cutting a pattern for the frame or strut assembly into the tubing generally is automated except for loading and unloading the length of tubing. For example, a pattern can be cut in tubing using a CNC-opposing collet fixture for axial rotation of the length of tubing, in conjunction with a CNC X/Y table to move the length of tubing axially relative to a machine-controlled laser as described. The entire space between collets can be patterned using a CO2 or Nd:YAG laser set-up. The program for control of the apparatus is dependent on the particular configuration used and the pattern to be ablated in the coding.

The frame could also be manufactured by laser cutting a large diameter tubing of Beta III titanium which would create the frame in its expanded position. Thereafter, the formed frame could be placed in its unexpanded position by backloading the frame into a restraining sheath which will keep the device in the unexpanded position until it is ready for use. If the frame is formed in this manner, there would be no need to heat treat the tubing to achieve the final desired diameter.

Frames for embolic filter devices are typically made from nitinol or stainless steel. The frames of the present invention are made from Beta titanium, and more particularly, Beta III titanium. Beta III titanium may be processed to have high yield strength and good ductility. The elastic modulus and spring-back values of Beta III titanium are between those of nitinol and stainless steel. The spring-back value of Beta III titanium is higher than for stainless steel and allows the frame to be cycled between its collapsed state and delivery state with minimal, or no, permanent deformation. Although the spring-back value of Beta III titanium is not as high as the spring-back value of superelastic nitinol, it is comparable to the spring-back value of linear pseudoelastic nitinol which is often used for embolic filter device frames.

The elastic modulus of Beta III titanium can be affected by processing and may be processed to values as high as 1.27×105 kg/cm² (1.8×106 lb/in²), which is about twice the elastic modulus of pseudoelastic nitinol. The higher elastic modulus is advantageous because it allows a reduction of the cross-sectional area of the frame while maintaining equal or nearly equal bending stiffness of the frame. For example, linear pseudoelastic nitinol material of the frame may be replaced with Beta III titanium of a smaller size, thereby reducing the overall collapsed profile of the device while maintaining equal or nearly equal bending stiffness.

Beta III titanium may be heat set to shape when forming the frame of the embolic filter device, which is an advantage over the mechanical bending process that is commonly used to process pseudoelastic nitinol. Mechanical bending is an involved process which requires expensive, complex tooling. Mechanical bending may involve multiple steps if a complex shape is being formed. Further, it is difficult to produce a consistent shape with mechanical bending unless the material is completely straight. With the ability to heat set shape of the Beta III titanium material, manufacturing costs for the frames of the embolic filter devices is decreased in comparison to frames made from nitinol or stainless steel because of a reduction in processing steps, a reduction in tooling costs, a reduction in operators and a reduction in time to produce the frames. The ability to heat set the Beta III titanium also eliminates variability between frames caused by imperfections' in the material, such as cast or helix in wire used to make the frame.

The polymeric material which can be utilized to create the filtering element includes, but is not limited to, polyurethane and Gortex™, a commercially available material. Other possible suitable materials include expanded polytetratluoroethylene (ePTFE). The material can be elastic or non-elastic. The wall thickness of the filtering element can be about 0.013-0.127 mm (0.00050-0.00500 inches). The wall thickness may vary depending on the particular material selected. The material can be made into a cone or similarly sized shape utilizing blow-mold technology. The openings can be any different shape or size. A laser, a heated rod or other process can be utilized to create perfusion openings in the filter material. The holes, would of course be properly sized to catch the particular size of embolic debris of interest. Holes can be lazed in a spiral pattern with some similar pattern which will aid in the re-wrapping of the media during closure of the device. Additionally, the filter material can have a “set” put in it much like the “set” used in dilatation balloons to make the filter element re-wrap more easily when placed in the collapsed position.

The material employed to manufacture the filtering element can be modified thermoplastic polyurethane elastomer. Such elastomers can be prepared by reacting polyester or polyester diol, a short-chain diol, a diisocyanate, and a substituted diol. The isocyanate portion is commonly referred to as the hard segment and the diol as the soft segment. It has been found that such a material offers excellent flexibility along with resistance to broad temperature ranges or tough end-use environments. Moreover, the presence of substituted diol makes the urethane non-blocking (non-sticking) and thus desirable in many medical applications including filtering and embolic protection systems use.

The filter element can be made from thermoplastic polyurethane elastomers (TPU) made with substituted “diol.” TPU's have both the mechanical as well as physical properties that are highly desirable in medical device applications. A filter element made with substituted “diol” TPU is non-blocking (non-sticking) and thus self adherence or undesirable adherence to other structures is minimized. Such a characteristic is a key to the effectiveness of a filter or other medical device as repeated manipulation and expansion and compression is common in the use of a filter. Thus, a filter made with modified TPU's (for example, modified Pellathane™), can consistently provide a surface or cavity for receiving matter and can be moved and expanded or contracted in vasculature to effectively accomplish its filtering function.

A combination of high tensile strength and high elongation of the modified thermoplastic polyurethane elastomers contemplated makes the material well-suited for dip forming or molding applications. Notably, conventional methods such as blow molding inherently create stresses and tensions in the element being blow molded. Where the element is a filter element, such stresses can make it difficult to couple the filter element to a frame or other structure by a melting process. Since dip forming or molding is a manufacturing option, the filter element can be made very thin.

In a preferred method, a solution of desirable filter material is mixed or formulated. A mandrel having the general shape and size of the filter frame or other medical device is dipped into the solution, removed and allowed to dry. The dipping and drying steps are repeated as necessary to create an element with desirable characteristics. Once the dip molding is completed, the element created is further processed for preparing the element to be coupled to a frame or medical device. The further processing involves removing any unwanted material or cutting openings in the element.

In certain applications, it may be desirable to apply a biocompatible lubricous coating to the filtering device. Such a lubricous coating can be Dow Corning 360 or other known biocompatible coatings. The coating can aid in the use of the filtering device, for example, by facilitating deployment and manipulation. The filter element itself can be coated as well as the frame or cage to which it is coupled.

The materials which can be utilized for the restraining sheath may include polymeric material, such as cross-linked high density polyethylene (HDPE). The sheath can alternatively be made from a material such as polyolifin which has sufficient strength to hold the compressed filtering assembly and has relatively low frictional characteristics to minimize any friction between the filtering assembly and the sheath. Friction can be further reduced by applying a coat of silicone lubricant, such as Microglide®, to the inside surface of the restraining sheath before the sheaths are placed over the filtering assembly.

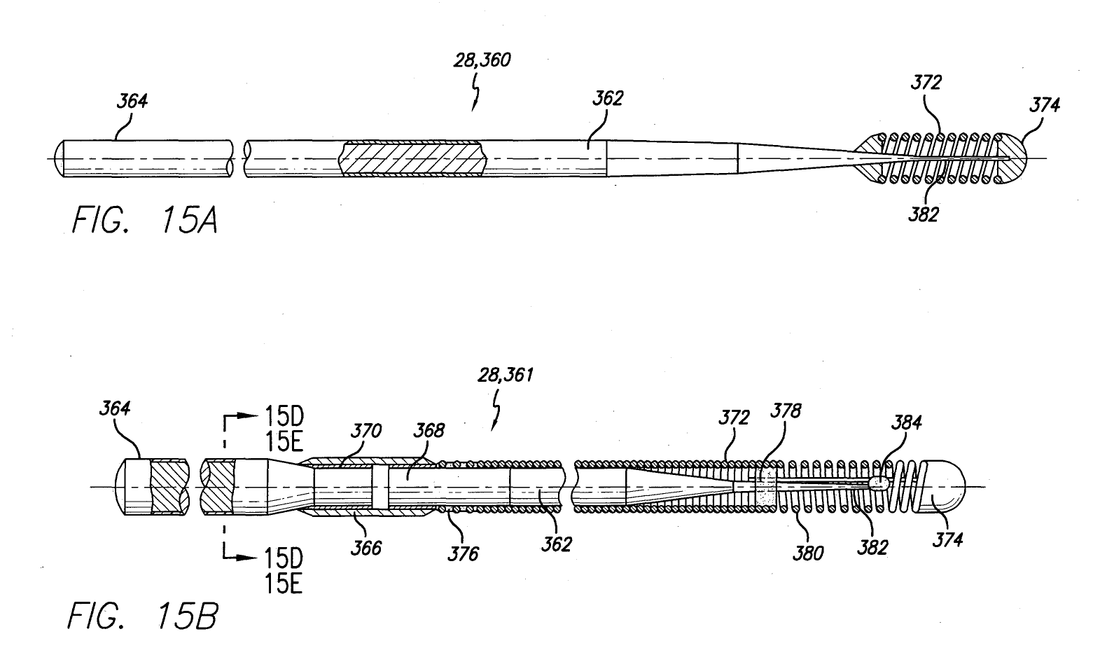

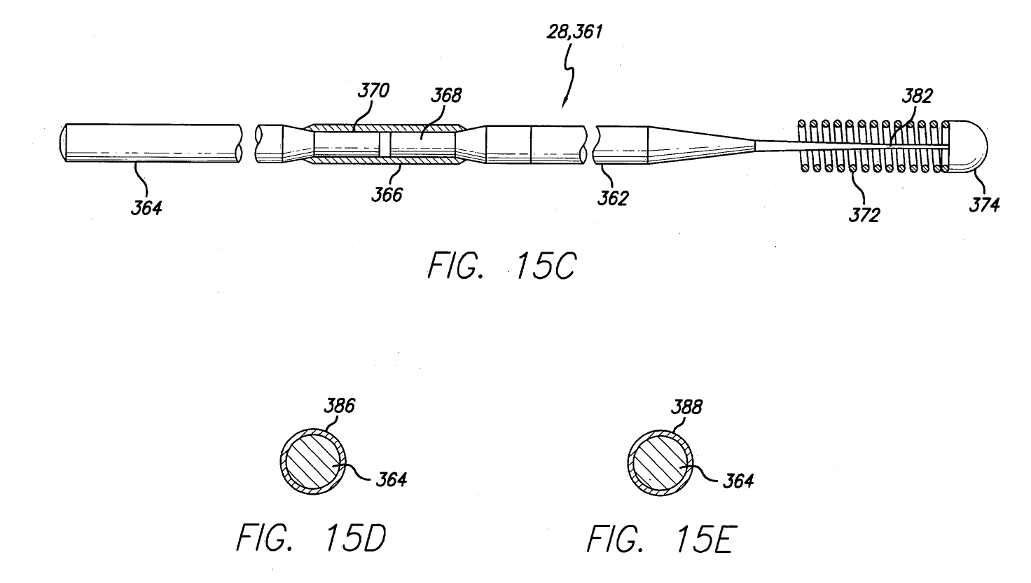

Referring to FIGS. 15A-15E, a guide wire 28 may include a core which is also made of Beta III titanium. The use of Beta III titanium for guide wires offers advantages over the use of other Beta titanium alloys, including higher spring-back, greater stiffness per cross-sectional area and better kink resistance. The kink resistance and stiffness of Beta III titanium lies between those of nitinol and stainless steel. More particularly, the kink resistance of Beta III titanium is higher than for stainless steel and the stiffness (columnar strength) of Beta III titanium is higher than for nitinol.

Beta III titanium may be included in the entire length of the guide wire core 360 (FIG. 15A), or may be used to make only a portion of the guide wire core 361, such as a distal section 362 or a proximal section 364 of the guide wire core (FIG. 15B). For example, in one embodiment of the invention the proximal section 364 of the guide wire core may be made of a first material, such as nitinol, which is coupled to the distal section 362 of the guide wire core which is made of a second material, such as Beta III titanium. The proximal section 364 and the distal section 362 may be coupled together in a torque transmitting relationship through methods that are well known in the art, such as by a connector element 366. Due to the properties of the Beta III titanium, if the diameters of the proximal 364 and distal 362 sections were equal (FIG. 15C), the distal section 362 of the guide wire core would be stiffer than the nitinol proximal section 364 of the guide wire core. Alternatively, the Beta III titanium distal section 362 of the guide wire core may be reduced in diameter (see FIG. 15B), thereby providing a low crossing profile while retaining stiffness through the distal section in comparison to the proximal section 364. In another embodiment of the invention, the distal section 362 has at least one tapered section that becomes smaller in the distal direction.

In one embodiment of the invention, the connector element 366 is preferably a hollow, tubular shaped structure having an inner lumen extending throughout the length of the connector element. The inner lumen of the connector element 366 is adapted to receive the proximal end 368 of the distal section 362 and the distal end 370 of the proximal section 364. The ends 368, 370 may be press fit into the connector element 366, or they may be secured therein by crimping, swaging the connector, or by means such as a suitable adhesive, weld, braze, or solder.

A helical coil 372 having a rounded plug 374 at the distal end thereof may be disposed about the distal section 362. The helical coil 372 is preferably secured to the distal section 362 at a proximal location 376 and at an intermediate location 378 by solder and by the distal end thereof to the rounded plug 374. Preferably, the most distal section 380 of the helical coil 372 is made of a radiopaque metal such as platinum, or platinum-nickel alloys to facilitate the identification thereof while it is disposed within a patient's body under a fluoroscope or x-ray. The most distal section 380 is preferably stretched about 10% to about 30% of the un-stretched length of the helical coil 372.

A most distal part 382 of the distal section 362 may optionally be flattened into a rectangular or square shaped cross-section and preferably provided with a rounded tip 384. The rounded tip 384 may be a bead of solder used to minimize the inadvertent passage of the most distal part 382 through the spacing between the stretched distal section 380 of the helical coil 372.

Another advantage of the use of Beta III titanium for the guide wire core is the ability to increase oxide 386 (see FIG. 15D) on the surface of the core, through heat treating or through environmental conditions, to increase lubricity of the core over that of a nitinol core. The oxide reduces the need of coatings, such as polymeric coatings, to attain a lubricious surface. However, if desired the exposed portion of the elongated proximal section 364 may be covered with a coating 388 (FIG. 15E) of a lubricious material such as polytetrafluoroethylene (sold under the trademark TEFLON) or other suitable lubricious coatings such as polysiloxane. A further benefit of the use of Beta III titanium for the guide wire 28 is the ability of the Beta III titanium to have its shape heat set. Stock Beta III titanium typically has a curve imposed on it due to the manufacturing process of making the Beta III titanium wire and the storage of the stock wire on spools. However, the desired shape of the wire may be heat set with good repeatability and low cost.

The particular embodiments of the aforementioned embolic filter devices and guide wires are not an exhaustive compilation of embolic filter devices and guide wires considered to be within the scope of the present invention, as the invention contemplates and includes the use of Beta titanium, and more particularly the use of Beta III titanium, for embolic filter devices and guide wires of other configurations. Further modifications and improvements may additionally be made to the devices and methods disclosed herein without departing from the scope of the present invention. Accordingly, it is not intended that the invention be limited, except as by the appended claims.

What is claimed:

1. A frame for an embolic filtering device used to capture embolic debris in a body vessel, the frame comprising:
a proximal strut assembly adapted to move between an unexpanded position and an expanded position; and
a distal strut assembly joined to the proximal strut assembly and adapted to move between an unexpanded position and an expanded position;
wherein the proximal strut assembly includes Beta titanium; and
wherein the distal strut assembly includes Beta titanium.

2. The frame of claim 1, wherein:
the Beta titanium of the proximal strut assembly is Beta III titanium; and
the Beta titanium of the distal strut assembly is Beta III titanium.

3. The frame of claim 2, further comprising a deployment ring, a distal end of the proximal strut assembly being coupled to the deployment ring, a proximal end of the distal strut assembly being coupled to the deployment ring, and the deployment ring configured to move between the unexpanded position and the expanded position.

4. The frame of claim 3, the proximal strut assembly including two self-expanding struts which move between the unexpanded position and the expanded position.

5. The frame of claim 3, the distal strut assembly including a plurality of self-expanding struts coupled to the deployment ring.

6. The frame of claim 5, further comprising a filtering element coupled to the distal strut assembly.

7. The frame of claim 3, further comprising a filtering element coupled to the deployment ring.

8. The frame of claim 3, wherein the proximal strut assembly includes a plurality of self-expanding struts which move between the unexpanded position and the expanded position, the deployment ring being coupled to a distal end of each of the self-expanding struts.

9. The frame of claim 8, wherein the deployment ring includes an undulating pattern of peaks and valleys.

10. The frame of claim 9, further comprising:
a filtering element coupled to the deployment ring, the filtering element having an opening for receiving embolic debris, the opening of the filtering element having the same undulating pattern as the deployment ring.

11. The frame of claim 2, further comprising a filtering element coupled to the distal strut assembly.

12. An embolic filtering device disposed on a guide wire and used to capture embolic debris in a body vessel, comprising:
an expandable filter assembly including a self-expanding frame having a proximal strut assembly, a distal strut assembly and a deployment ring disposed between and coupling the proximal strut assembly and the distal strut assembly, the distal strut assembly having a filter element coupled thereto, the self-expanding frame including Beta titanium, the expandable filter assembly being adapted to move between an unexpanded position and an expanded position; and
means for mounting the expandable filter assembly to the guide wire.

13. The embolic filtering device of claim 12, wherein the Beta titanium of the self-expanding frame being Beta III titanium.

14. The filtering device Of claim 13, further including means for maintaining the filter assembly in the unexpanded position until it is ready to be deployed into the expanded position.

15. An expandable frame for an embolic filtering device, comprising:
a first half frame adapted to move between an unexpanded position and an expanded position, the first half frame having a first control arm connected to a second control arm by a partial loop, the first half frame including Beta titanium; and
a second half frame adapted to move between an unexpanded position and an expanded position, the second half frame having a first control arm connected to a second control arm by a partial loop, the second half frame including Beta titanium;
wherein the partial loops of the first and second half frames cooperate to form a composite opening for coupling of a filtering element when placed in the expanded position.

16. The expandable frame of claim 15, wherein:
the Beta titanium of the first half frame is Beta III titanium; and
the Beta titanium of the second half frame is Beta III titanium.

17. An embolic filtering device, comprising:
an elongated member; and
a self-expanding frame, having,
a first half frame adapted to move between an unexpanded position and an expanded position, the first half frame having a first control arm connected to a second control arm by a partial loop, the first half frame including Beta titanium, and
a second half frame adapted to move between an unexpanded position and an expanded position, the second half frame having a first control arm connected to a second control arm by a partial loop, the second half frame including Beta titanium,
wherein the partial loops of the first and second half frames cooperate to form a composite opening;
a filtering element coupled to the partial loops of the first and second half frames; and
a filter support structure upon which the expandable filter assembly is mounted, the filter support structure having a lumen extending therethrough to receive the elongated member.

18. The embolic filtering device of claim 17, wherein:
the Beta titanium of the first half frame is Beta III titanium; and
the Beta titanium of the second half frame is Beta III titanium.

19. The embolic filtering device of claim 18, further including means for rotatably mounting the expandable filter assembly to the elongated member.

20. The embolic filtering device of claim 18, wherein the filter support structure is a coil.

21. An expandable frame for an embolic filtering device, comprising:
a first control arm having a distal end and a proximal end;
a second control arm having a distal end and a proximal end; and
a pair of partial loops connected to the first and second control arms near the distal ends of the first and second control arms, the partial loops being adapted to move between an unexpanded position and an expanded position, the partial loops cooperating to form a composite loop when placed in the expanded position, the partial loops being adapted for coupling of a filtering element;
wherein the expandable frame includes Beta titanium.

22. The expandable frame of claim 21, wherein the Beta titanium of the expandable frame is Beta III titanium.

23. The expandable frame of claim 99, wherein the frame includes strain distributing struts to increase the bendability of the frame.

24. The expandable frame of claim 23, wherein the strain distributing struts are located on the partial loops near the point of coupling to the distal ends of the first and second control arms.

25. The expandable frame of claim 93, wherein the strain distributing struts are formed on the frame and have a thinner width than the remainder of the strut forming the frame.

26. The expandable frame of claim 22, further including a second set of distal control arms coupled to the frame and extending distally away from the frame.

27. An embolic filtering system, comprising:
an expandable filter assembly including a self-expanding frame moveable between an expanded position and an unexpanded position, the expandable filter assembly being made of Beta titanium;
a filtering element coupled to and movable with the expandable filter; and
a restraining sheath having a distal end portion and a proximal end, the distal end portion of the restraining sheath being adapted to receive the expandable filter assembly for maintaining the filter assembly in the unexpanded position and being movable to expose the filter assembly.

28. The embolic filtering system of claim 27, wherein the Beta titanium of the expandable filter assembly is Beta III titanium.

29. An embolic filtering device, comprising:
a frame adapted to move between an unexpanded position and an expanded position, the frame being made of Beta titanium; and
a filter element coupled to the frame.

30. The embolic filtering device of claim 29, wherein the Beta titanium of the frame is Beta III titanium.

31. An elongated guide wire, comprising:
an elongated core having proximal and distal core sections; and
a flexible body disposed about and secured to the distal core section;
wherein the core includes Beta III titanium.

32. The guide wire of claim 31, wherein the guide wire core including the proximal and distal sections is uninterrupted and the proximal and distal sections include the Beta III titanium.

33. The guide wire of claim 31, wherein the distal section of the guide wire core includes Beta III titanium.

34. The guide wire of claim 33, wherein the distal section of the guide wire core includes a diameter smaller than the diameter of a proximal section of the guide wire core.

35. The guide wire of claim 34, wherein the flexible body includes at least one helical coil.

36. The guide wire of claim 31, wherein the proximal section of the guide wire core includes Beta III titanium.

37. The guide wire of claim 31, wherein the Beta III titanium core includes a shape which is heat set into the Beta III titanium core.

38. The guide wire of claim 31, further comprising a torque-transmitting tube joining the proximal and distal core sections together.
